(19) United States
(12) Patent Application Publication
(10) Pub. No.: US 2003/0180882 A1
(43) Pub. Date: Sep. 25, 2003

(54) Expression of proteolytically-sensitive peptides

(76) Inventors: William E. Courchesne, Soda Springs, CA (US); David A. Schooley, Reno, NV (US); Kathrin Copley, San Diego, CA (US)

Correspondence Address: Reza Green, Esq., Novo Nordisk Pharmaceuticals, Inc., 100 College Road West, Princeton, NJ, 08540, US

(21) Appl. No.: 10/278,242

(22) Filed: Oct. 23, 2002

Related U.S. Application Data
(63) Continuation of application No. 09/661,452, filed on Sep. 13, 2000, now abandoned.
Application No. 09/661,452 is a continuation of application No. 09/237,936, filed on Jan. 27, 1999, now abandoned.

(51) Int. Cl. C12P 21/02; C07H 21/04; C12N 1/18; C12N 9/50; C12N 15/74; C07K 14/39

(52) U.S. Cl. 435/69.1; 435/219; 435/254.2; 435/320.1; 536/23.2; 435/483; 530/350

(57) ABSTRACT

Compositions and methods are disclosed for the production of polypeptides sensitive to proteolysis due to their content of arginine and lysine residues. The methods of the invention utilize yeast cells with reduced expression of either or both of the proteases encoded by YAP3 and MKC7. The methods of the invention also utilize yeast cells with reduced activity for either or both of the Yap3 and Mkc7 proteases.

While a yeast expression system is used to demonstrate the invention, any eukaryotic cell which normally has homologues to the Yap3 and Mkc7 endoproteases may be used in the disclosed compositions and methods.

### RELATED APPLICATIONS

This application claims priority to U.S. Provisional Application 60/072,691 filed on Jan. 27, 1998, which is herein incorporated by reference in its entirety.

### STATEMENT OF RIGHTS TO INVENTIONS MADE UNDER FEDERALLY SPONSORED RESEARCH

This invention was partially made with support from the Nevada Agricultural Experiment Station and under NIH grant number GM 48172.

### FIELD OF THE INVENTION

The present invention relates to compositions and methods for the expression of peptides in eukaryotic cells, wherein such peptides are sensitive to proteolysis due to their content of arginine and lysine residues.

The present invention is based on the unexpected discovery that the successful expression of intact peptides is made possible by using cells for the expression system which have reduced expression of either or both of the proteases encoded by YAP3 and MKC7. The methods and compositions of the present invention greatly increase the yield of intact recombinant peptides when these peptides contain Arg and/or Lys residues.

For the ease of isolation and purification of these peptides, a synthetic gene encoding a precursor of the heterologous peptide is constructed containing a signal peptide, such as the Saccharomyces cerevisiae α-factor prepro sequence, to direct secretion of the peptide from the yeast cell.

Related methods and compositions can be used for the heterologous expression of various peptides from a wide variety of eukaryotic cell types in addition to yeast cells.

### BACKGROUND OF THE INVENTION

Overview.

All publications and patent applications herein are incorporated by reference to the same extent as if each individual publication or patent application was specifically and individually indicated to be incorporated by reference.

Cloning a gene or cDNA encoding a particular protein is only the first of many steps needed to produce a recombinant protein for medical or industrial use. The next step is to put the gene into a host cell for production.

The most popular expression systems for protein production include the bacteria Escherichia coli and Bacillus subtilis, yeast (the most important of which is Saccharomyces cerevisiae), cultured mammalian cells and baculovirus-infected insect cells. For detailed descriptions of the various expression systems and the methods for their use in producing proteins, see, e.g., Fernandez et al., Gene Expression Systems (1998, Academic Press); Jones et al., Vectors: Expression Systems: Essential Techniques (1998, Essential Techniques Series); Brown, Gene Cloning: An Introduction, Second Edition (1990, Chapman & Hall); Esposito, Yeast Molecular Biology—Recombinant DNA: Recent Advances (1984, Berkeley Workshop); Glover and Hames, DNA Cloning 2: Expression Systems (1995, Practical Approach Series 149, Volume 2).

Important considerations for the production of biologically active peptides include the desire for high levels of expression of the recombinant peptide, the requirement for proper post-translational modification, secretion of peptide for easier purification, and proper processing from a precursor form at the N-terminus.

Yeast Endoproteases.

Yeast is a simple eukaryote that resembles mammalian cells in many ways but can be grown as quickly and cheaply as bacteria can. Yeast perform many of the posttranslational modifications found on human proteins and can be induced to secrete certain proteins into the growth medium for harvesting. A disadvantage of yeast is the presence of active proteases that degrade foreign proteins, thereby reducing the yield of the product.

Kex2 endoprotease of Saccharomyces yeast is a protease which specifically processes a mating type factor and a killer particle (Leibowitz and Wickner, 1976, PNAS USA 2061-2065; Julius et al., 1984, Cell 37:1075-1089). Normal Kex2 and various truncated forms of Kex2 have been used to produce biologically active polypeptides, the activation of which requires post-translational processing (U.S. Pat. Nos. 5,162,220; 5,234,830 and 5,521,093).

Yeast cells express an alternate enzyme encoded by the Yeast Aspartyl Protease 3 (YAP3) gene which can process pro-alpha-mating factor when this pheromone is overexpressed in Kex2-deficient mutants (Azaryan et al., 1993, J. Biol. Chem. 268(16):11968-11975). The yeast Kex2 and Yap3 endoproteases have distinct, though overlapping, substrate specificities (Bourbonnais et al., 1994, Biochimie 76(3-4):226-233; Chaudhuri et al., 1995, FEBS Lett. 364(1):91-97). Yap3 contains a C-terminal serine/threonine-rich sequence and potential transmembrane domain similar to those found in the KEX2 gene product (Egel-Mitani et al., 1990, Yeast 6(2):127-137). The complete sequence of YAP3 can be found at GenBank Accession No. L31651.

More recently, a third endopeptidase gene, MKC7, with considerable homology to YAP3 has been detected (Komano and Fuller, 1995, Proc. Natl. Acad. Sci. USA, 92:10752-10756). The complete sequence of MKC7 can be found at GenBank Accession No. U14733. While disruption of MKC7 or YAP3 alone resulted in no observable phenotype, impaired growth at 37° C. was observed for mkc7yap3 double disruptants (Id.).

Limitations of Current Polypeptide Production Methods.

Some peptides less than 100 amino acids in length have been successfully expressed and secreted in yeast-based expression systems; however, the majority of these peptides are heavily constrained with disulphide bonds. Peptides with no disulphide bonds have been difficult to express without significant proteolysis.

We describe novel methods of producing and secreting peptides by eukaryotic cells, wherein the methods utilize mutant cells lacking Mkc7 and/or Yap3 proteases. Our methods result in a significantly higher expression of the desired peptide over the expression levels obtained using previously known methods. Although yeast cells are used in a majority of the experiments, the expression systems and methods of the present invention are extendable to use with any eukaryotic cells, including, for example, insect, vertebrate and plant cells.

### SUMMARY OF THE INVENTION

The present invention provides compositions and methods for the production of polypeptides sensitive to proteolysis, wherein the sensitivity to proteolysis is due to the content of arginine and lysine residues in the polypeptide. The methods of the present invention are particularly useful for the production of heterologous polypeptides.

The present invention provides methods useful for the production of polypeptides sensitive to proteolysis due to the content of arginine and lysine residues in the polypeptides wherein the methods comprise: (1) providing a yeast cell with reduced expression of either or both of the proteases encoded by YAP3 and MKC7; (2) transforming the yeast cell with a vector comprising a yeast promoter operably linked to a DNA sequence coding for the polypeptides sensitive to proteolysis; (3) expressing the polypeptides; and (4) isolating the polypeptides.

The methods of the present invention involve reducing the expression of either or both of the proteases encoded by YAP3 and MKC7. The reduction in expression can be accomplished by any effective process including: (1) elimination of either or both of the proteases encoded by YAP3 and MKC7; (2) disabling either or both of the proteases encoded by YAP3 and MKC7;

and (3) antisense binding.

The present invention further provides methods useful for the production of polypeptides sensitive to proteolysis due to their content of arginine and lysine residues, wherein the methods comprise the steps of: (1) providing a yeast cell with reduced activity for either or both of the Yap3 and Mkc7 proteases; (2) transformning the yeast cell with a vector comprising a yeast promoter operably linked to a DNA sequence coding for the polypeptide sensitive to proteolysis;

(3) expressing the polypeptide; and (4) isolating the polypeptide.

The present invention further provides methods for the production of polypeptides sensitive to proteolysis due to their content of arginine and lysine residues, wherein the methods comprise the steps of: (1) providing a eukaryotic cell with reduced expression of either or both of the proteases encoded by YAP3 and MKC7; (2) transforming the eukaryotic cell with a vector comprising a promoter operably linked to a DNA sequence coding for the polypeptides sensitive to proteolysis; (3) expressing the polypeptides; and (4) isolating the polypeptides.

The present invention further provides methods for the production of polypeptides sensitive to proteolysis due to their content of arginine and lysine residues, wherein the methods comprise the steps of: (1) providing a eukaryotic cell with reduced activity for either or both of the Yap3 and Mkc7 proteases; (2) transforming the eukaryotic cell with a vector comprising a promoter operably linked to a DNA sequence coding for the polypeptides sensitive to proteolysis; (3) expressing the polypeptides; and (4) isolating the polypeptides.

The present invention further provides such methods of producing proteolytically sensitive polypeptides wherein the vectors used in the methods further comprise a signal peptides to direct secretion of the polypeptides from the yeast cell.

Furthermore, the present invention further provides such methods of producing proteolytically sensitive polypeptides wherein the signal peptide is Saccharomyces cerevisiae α-factor prepro sequence.

Even furthermore, the present invention further provides such methods of producing proteolytically sensitive polypeptides wherein the polypeptide further comprises a glycine residue at its carboxyl terminus.

Even furthermore, the present invention further provides such methods of producing proteolytically sensitive polypeptides wherein the polypeptides are treated with peptidyl-glycine amidating enzyme after isolating the polypeptides.

The methods of the present invention are useful for producing any proteolytic-sensitive peptide as described herein, including a precursor of Manduca sexta diuretic hormone, a site-directed mutant M. sexta diuretic hormone, albumin, human parathyroid hormone, insulin, glicentine related polypeptide, glucagon, calcitonin, neuropeptide Y, and analogues of each of these polypeptides.

The methods of the present invention can be accomplished using any eukaryotic cell, including insect cells, plant cells and mammalian cells.

Further objects and advantages of the present invention will be clear from the description that follows.

### BRIEF DESCRIPTION OF THE DRAWINGS

FIG. 1. Diagram of the pKSC2 Expression Vector.

View of the Mas-DH gene. The elements in the diagram are not drawn to scale. The synthetic 168 base oligonucleotide coding for the peptide linker and Mas-DH+Gly coding region is set forth in Example 1. The Gly is required for post-translational amidation.

FIG. 2. RPLC Chromatograms of Mas-DH+Gly Fragments Recovered from a Series of Isogeneic Strains Transformed with pKSC2.

RPLC conditions were similar for A, B, and C. Proteins were eluted at 1 ml/min using a linear gradient of 2% to 60% CH₃CN-0.1% TFA in 60 min. Peaks were collected by hand and analyzed by ESI-MS. RPLC conditions for D are reported in RPLC purification.

Proteins were eluted at 5 ml/min using a linear gradient of 10% to 60% CH₃CN-0.1% TFA in 50 min. Peaks were collected and analyzed by ESI-MS. For all figures absorbance was measured at 220 nm.

In FIGS. A, B, C and D the Mas-DH+Gly fragments are labeled as follows: [1-22] corresponds to the fragment Mas-DH[1-22]; [1-21], Mas-DH[1-21]; [15-42], Mas-DH+Gly[15-42]; [23-42], Mas-DH+Gly[23-42]; [25-42], Mas-DH+Gly[25-42]. The peak marked by the asterisk (*) was present in cells not expressing Mas-DH+Gly. The molecular weights of unlabeled peaks do not correspond to the calculated molecular weights of fragments of Mas-DH+Gly. Extinction coefficients at 220 nm are sequence-dependent and cannot be readily used for a weight or nanomole-based relative quantification.

FIG. 3. RPLC Chromatograms of Mas-DH[K22Q]+Gly Fragments Recovered from a Series of Isogeneic Strains Transformed with pKSC3.

RPLC conditions were similar for A, B, C and D. All peaks were collected and analyzed by ESI-MS. For all figures absorbance was measured at 220 nm.

In FIGS. A, B, C and D the Mas-DH[K22Q]+Gly fragments are labeled as follows: [1-24] corresponds to the fragment Mas-DH[K22Q][1-24]; [15-42], Mas-DH[K22Q]+Gly[15-42]; [25-42], Mas-DH[K22Q]+Gly[25-42]; [26-42], Mas-DH[K22Q]+Gly[26-42]. Full-length Mas-DH[K22Q]+Gly is labeled as DH. The peak marked by the asterisk (*) was present in cells not expressing Mas-DH[K22Q]+Gly. The molecular weights of unlabeled peaks do not correspond to the calculated molecular weights of fragments of Mas-DH[K22Q]+Gly.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Protease Inhibitors

Proteinase inhibitors can be utilized to prevent proteolysis by proteinases. By using appropriate inhibitors, proteinases can be inactivated, either reversibly or irreversibly, in a preparation. Although variation in proteinases exists between organisms, there is only a small number of types of proteinases. There is also considerable overlap in the ways in which proteolysis in different systems can be controlled.

Examples of specific proteinase inhibitors include those for the following types of proteinases: aspartic (e.g., Pepstatin A), cysteine (e.g., iodoacetic acid), cysteine/serine (e.g., PMSF also known as phenylmethanesulphonyl fluoride), serine (e.g., benzamidine), metallo-(e.g., EDTA) and calpains (e.g., chelating agents).

For additional proteinase inhibitors and the preparation and use of said inhibitors, see, for example, R. J. Beynon & J. S. Bond (Editors), 1989, Proteolytic Enzymes: A Practical Approach (IRL Press); Takada, A. (Editor), 1989, Proceedings of the 2nd International Conference on Fibronolysis, Hamamatsu, Japan 26th August 1989 (International Congress Series); Murano, Genesio, 1985, Protease Inhibitors of Human Plasma Biochemistry and Pathophysiology; Cheronis, et al., 1993, Proteases, Protease Inhibitors and Protease-Derived Peptides: Importance in Human Pathophysiology and Therapeutics (Agents and Actions Supplements); 8th Winter School on Proteinases and Their Inhibitors: recent developments, 1989 (Tiers, Mar. 8-12, 1989); Barrett et al., 1987, Proteinase Inhibitors (Research Monographs in Cell Tissue Physiology, Vol. 12).

Recombinant DNA

In accordance with the present invention, as described above or as discussed in the Examples below, there may be employed conventional molecular biology, microbiology and recombinant DNA techniques. Such techniques are explained fully in the literature. See for example, Sambrook et al., Molecular Cloning: A Laboratory Manual (Second Ed., Cold Spring Harbor Press, Cold Spring Harbor N.Y., 1989); DNA Cloning: A Practical Approach, vol. 1 and 2 (D. N. Glover ed., 1985); Oligonucleotide Synthesis (Transcription and Translation M. J. Gait ed., 1984); Nucleic Acid Hybridization (B. D. Hames et al., 1985); (B. D. Hames et al., eds, 1984); E. Harlow et al., Antibodies: A Laboratory Manual (Cold Spring Harbor Press, Cold Spring Harbor N.Y., 1988); Roe et al., DNA Isolation and Sequencing: Essential Techniques (John Wiley & Sons, NY, 1996) and Ausubel et. al., Current Protocols in Molecular Biology (Greene Publishing Co. NY, 1995) to name a few.

Disabling Genes.

An example of an effective disabling modification would be a single nucleotide deletion occurring at the beginning of a gene that would produce a translational reading frameshift. Such a frameshift would disable the gene, resulting in non-expressible gene product and thereby disrupting functional protein production by that gene. Protease production by the gene could be disrupted if the regulatory regions or the coding regions of the protease genes are disrupted.

In addition to disabling genes by deleting nucleotides, causing a transitional reading frameshift, disabling modifications would also be possible by other techniques including insertions, substitutions, inversions or transversions of nucleotides within the gene's DNA that would effectively prevent the formation of the protein coded for by the DNA.

It is also within the capabilities of one skilled in the art to disable genes by the use of less specific methods. Examples of less specific methods would be the use of chemical mutagens such as hydroxylamine or nitrosoguanidine or the use of radiation mutagens such as gamma radiation or ultraviolet radiation to randomly mutate genes. Such mutated strains could, by chance, contain disabled YAP3 and MKC7 genes such that the genes were no longer capable of producing functional aspartyl proteases. The presence of the desired disabled genes could be detected by routine screening techniques. For further guidance, see U.S. Pat. No. 5,759,538.

Antisense RNA

Antisense molecules are RNA or single-stranded DNA molecules with nucleotide sequences complementary to a specified mRNA. When a laboratory-prepared antisense molecule is injected into cells containing the normal mRNA transcribed by a gene under study, the antisense molecule can base-pair with the mRNA, preventing translation of the mRNA into protein. The resulting double-stranded RNA or RNA/DNA is digested by enzymes that specifically attach to such molecules. Therefore, a depletion of the mRNA occurs, blocking the translation of the gene product so that antisense molecules find uses in medicine to block the production of deleterious proteins.

Methods of producing and utilizing antisense RNA are well known to those of ordinary skill in the art (see, for example, C. Lichtenstein and W. Nellen (Editors), Antisense Technology: A Practical Approach, Oxford University Press (December, 1997); S. Agrawal and S. T. Crooke, Antisense Research and Application (Handbook of Experimental Pharmacology, Volume 131), Springer Verlag (April, 1998); I. Gibson, Antisense and Ribozyme Methodology: Laboratory Companion, Chapman & Hall (June, 1997); J. N. M. Mol and A. R. Van Der Krol, Antisense Nucleic Acids and Proteins, Marcel Dekker; B. Weiss, Antisense Oligodeoxynucleotides and Antisense RNA: Novel Pharmacological and Therapeutic Agents, CRC Press (June, 1997); Stanley et al., Antisense Research and Applications, CRC Press (June, 1993); C. A. Stein and A. M. Krieg, Applied Antisense Oligonucletotide Technology (April, 1998)).

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding Yap3 and Mkc7. Such DNA sequences may be incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept can be extended by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Delivery of Nucleic Acids

Recombinant methods known in the art can be used to achieve the antisense or triplex inhibition of a target nucleic acid. For example, vectors containing antisense nucleic acids can be employed to express protein or antisense message to reduce the expression of the target nucleic acid and therefore its activity. Such vectors are known or can be constructed by those skilled in the art and should contain all expression elements necessary to achieve the desired transcription of the antisense or triplex sequences. Other beneficial characteristics can also be contained within the vectors such as mechanisms for recovery of the nucleic acids in a different form.

Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids, liposomes and other recombination vectors.

The vectors can also contain elements for use in either procaryotic or eukaryotic host systems. One of ordinary skill in the art will know which host systems are compatible with a particular vector.

The vectors can be introduced into cells or tissues by any one of a variety of known methods within the art. Such methods can be found described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), and include, for example, stable or transient transfection, lipofection, electroporation and infection with recombinant viral vectors. Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the antisense vectors to specific cell types in vivo or within a tissue or mixed culture of cells. Viral vectors can also be modified with specific receptors or ligands to alter target specificity through receptor mediated events.

Knock-outs and Knock-ins

The development of transgenic technology allows investigators to create eukaryotic organisms of virtually any genotype and to assess the consequences of introducing specific foreign nucleic acid sequences on the physiological and morphological characteristics of the transformed animals. The availability of transgenics permits cellular processes to be influenced and examined in a systematic and specific manner not achievable with most other test systems. For example, the development of transgenic animals provides biological and medical scientists with models that are useful in the study of disease. Such animals are also useful for the testing and development of new pharmaceutically active substances.

Transgenic eukaryotes can be produced by a variety of different methods including transfection, electroporation, microinjection, gene targeting in embryonic stem cells and recombinant viral and retroviral infection (see, e.g., U.S. Pat. Nos. 4,736,866; 5,602,307; Mullins et al. (1993) Hypertension 22(4):630-633; Brenin et al. (1997) Surg. Oncol. 6(2)99-110; Tuan (ed.), Recombinant Gene Expression Protocols, Methods in Molecular Biology No. 62, Humana Press (1997)).

The term “knock-out” generally refers to mutant organisms which contain a null or non-functional allele of a specific gene. The genes which are knocked-out are inactivated or disrupted. Therefore, knock-out organisms do not produce the polypeptide normally coded for by the non-knock-out gene in wildtype organisms. For examples of producing knock-out organisms, see, e.g., U.S. Pat. Nos. 5,807,995 and 5,849,991.

The term “knock-in” generally refers to mutant organisms into which a gene has been inserted through homologous recombination. The knock-in gene may be a mutant form of a gene which replaces the endogenous, wild-type gene. Such mutations include insertions of heterologous sequences, deletions, frameshift mutations and any other mutations that prevent, disrupt or alter normal gene expression.

Transgenic procedures have been successfully utilized in a variety of murine and non-murine animals, including sheep, goats, pigs, dogs, cats, monkeys, chimpanzees, hamsters, rabbits, cows and guinea pigs (see, e.g., U.S. Pat. Nos. 4,736,866; 5,728,915; 5,731,490; Kim et al., Mol. Reprod. Dev. 46(4):515-526 (1997); Houdebine, Reprod. Nutr. Dev. 35(6):609-617 (1995); Clutter et al., Genetics 143(4):1753-1760 (1996); Petters, Reprod. Fertil. Dev. 6(5):643-645 (1994); Schnieke et al., Science 278(5346):2130-2133 (1997); Amoah, Animal Science 75(2):578-585(1997); McCarthy, The Lancet 349(9049):405 (1997)).

Methods of producing transgenic plants are also well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and

Agrobacterium-mediated transformation (see, e.g., U.S. Pat. Nos. 5,405,765, 5,472,869, 5,538,877, 5,538,880, 5,550,318, 5,641,664, 5,736,369 and 5,736,369; Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990);

and, Raineri et al., Bio/Tech. 8:33-38 (1990)).

Other methods that can be used to inhibit expression of an endogenous gene may also be used in the present methods. For example, formation of a triple helix at an essential region of a duplex gene serves this purpose. The triplex code, permitting design of the proper single stranded participant is also known in the art. (See H. E. Moser, et al., Science 238: 645-650 (1987) and M. Cooney, et al., Science 241: 456-459 (1988)). Regions in the control sequences containing stretches of purine bases are particularly attractive targets. Triple helix formation along with photocrosslinking is described, e.g., in D. Praseuth, et al., Proc. Nat'l Acad. Sci. USA 85: 1,349-1,353 (1988).

Isolating Homologues to Yap3 and Mkc7

Essentially, a skilled artisan can readily use the amino acid sequence of Yap3 (GenBank Accession Number L31651) or Mkc7 (GenBank Accession Number U14733) to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gtll library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism.

Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Probes to detect differences in RNA expression levels between cells exposed to the agent and control cells may be prepared from the nucleic acids of the genes utilized in the present invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target'sequence. Methods to optimize probe specificity are commonly available in Sambrook et al. (Molecular Cloning: A Laboratory Approach, Cold Spring Harbor Press, NY, 1989) or Ausubel et al. (Current Protocols in Molecular Biology, Greene Publishing Co., NY, 1995).

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the protein having the sequence of Yap3 or Mkc7 are identified.

Hybridization for qualitative and quantitative analysis of mRNAs may also be carried out by using a RNase Protection Assay (i.e., RPA, see Ma et al. (1996) Methods 10: 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes, pH 6.4, 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

The specific examples presented below are illustrative only and are not intended to limit the scope of the invention.

### EXAMPLES

### Introduction to Examples 1-7

Two Saccharomyces cerevisiae genes, YAP3 (Egel-Mitani et al., 1990) and MKC7 (Komano & Fuller, 1995), were identified by their abilities to suppress certain abnormal phenotypes associated with loss of Kex2 activity in mutant cells (i.e., cells with a kex2Δ gene deletion). Both YAP3 and MKC7 encode homologous aspartyl proteases (with the same name as the gene), which have limited homology to aspartyl proteases from other organisms, including humans (Komano & Fuller, 1995).

The functional homology between Kex2 and mammalian protein precursor processing enzymes, such as furin, is well established (Steiner et al., 1992). Such enzymes are implicated in peptide hormone, neuropeptide, viral envelope glycoprotein, growth factor, and growth factor receptor precursor processing. The demonstrated similarity between Kex2-mediated precursor processing in yeast and other organisms establishes the validity of using yeast protein precursor processing in general as a model for other organisms.

It is a reasonable assumption, therefore, that the recently identified Yap3 and Mkc7 endoproteases will also have closely related homologues with similar functions in other organisms.

Zhang et al. (1997, Biochim. Biophys. Acta 1359(2):110-122) found that deletions of yap3 and mkc7 decreased alpha-secretase activity by 56 and 29%, respectively; whereas, the double deletion decreased the activity by 86%. They suggested that the Yap3 and Mkc7 proteases should be investigated as candidate secretases in mammalian tissues.

The compositions and methods of the present invention require the disruption or elimination of the genes coding for Yap3 and/or Mkc7.

As an example of the utility of the present invention, we give precise conditions for the production of a 42 amino acid precursor of the 41 amino acid diuretic hormone (DH) of the insect Manduca sexta. This peptide is an especially difficult test of the instant methodology as it contains 3 Lys and 5 Arg residues and no disulfide bonds. This peptide is also a good test for our expression system because it is a natural peptide that normally survives an insect cell secretory pathway, and the degradation products can be isolated and characterized using Reversed-Phase Liquid Chromatography (RPLC) and electrospray ionization mass spectrometry (ESI-MS).

The precursor differs from the DH in having an extra Gly residue at the C-terminus. After isolation of the secreted 42 amino acid precursor, the DH is formed by treating the precursor with peptidylglycine amidating mono-oxygenase (PAM), a bifunctional enzyme found in the brain of the insect M. sexta (Bernasconi et al., 1992) and many vertebrates (Eipper et al., 1992). PAM oxidizes the α carbon of the glycine, resulting in addition of a hydroxyl moiety on this carbon. A lyase activity of the enzyme then cleaves the oxidized precursor to the mature DH, containing an amidated carboxyl terminus, and forming glyoxalate from the carbons of the glycine. The PAM enzyme is commercially available (Unigene®).

As with many bioactive peptides; the α-amidation is crucial for biological activity—synthetic Mas-DH with a C-terminal acidic residue has 1000-fold reduced biological activity (Audsley et al., 1995).

Successful expression of full-length Mas-DH+Gly required the use of protease deficient yeast strains. In wild-type strains, Mas-DH+Gly was recovered only as proteolytic fragments, even in the presence of various protease inhibitors. Expression of Mas-DH+Gly in strains deficient in either the Mkc7 or the Yap3 protease reduced proteolysis, while no proteolysis of Mas-DH+Gly was detectable in a strain lacking both proteases. Analysis of recovered proteolytic fragments from strains other than the double mutant claimed in this invention (including a pep4 strain deficient in vacuolar proteases) revealed a complex pattern of cleavage sites. Analysis of many cleavage sites showed that Yap3 preferred to cleave after either Lys or Arg and Mkc7 after Lys in vivo.

Because of the proteolysis observed with non-mutant strains, we examined the literature for peptides less than 10 kDa in size which have been expressed and secreted from S. cerevisiae. A list of such peptides is shown in Table 1. Peptides which were expressed as part of a fusion protein requiring further processing and purification are not listed in the table.

TABLE 1
Heterologous Expression of secreted peptides <10 kDa in S. cerevisiae

| | No. of | No. of | No. of | Cleav- | |
|---|---|---|---|---|---|
| | amino | disulfide | Lys and | age | |
| Peptide Name | acids | bonds | Arg | sites | Production |
| Somatostatin (Bourbonnais et al., 1988) | 14 | 1 | 2 | | full-length² |
| Glucagon (Moody et al., 1987) | 29 | 0 | 3 | R18 | evidence of cleavage; products |
| | | | | | uncharactenzed |
| β-endorphin (Bitter et al, 1984) | 31 | 0 | 5 | K9, | no full-length product found |
| | | | | K19 | |
| calcitonin (Zsebo et al., 1986) | 32 | 1 | 1 | | detection by RIA |
| scorpion insect-selective toxin I5A (Pang et | 35 | 4 | 5 | | not active |
| al., 1992) | | | | | |
| Antifreeze peptide AFP6 (Driedonks et al., | 37 | 0 | 2 | | required multicopy gene for |
| 1995) | | | | | expression |
| Mas-DH + Gly | 42 | 0 | 8 | | full-length³ |
| Echistatin (Jacobson et al., 1989) | 49 | 4 | 7 | R22 | ˜93% full-length, ˜7% cleavage |
| hEGF (urogastrone)¹ (Urdea et al., 1983) | 53 | 3 | 5 | | full-length² |
| h pancreatic secretory trypsin inhibitor¹ | 55 | 3 | 7 | | full-length² |
| (Izumoto t al., 1987) | | | | | |
| insulin (Thim et al., 1987) | 55 | 3 | 2 | | full-length² |
| Aprotinin (Norris et al., 1990) | 58 | 3 | 10 | R42 | some cleavage, K41S mutation |
| | | | | | increased yield |
| Bovine pancreatic trypsin inhibitor | 58 | 3 | 10 | | full-length² |
| (Driedonks et al., 1995) | | | | | |
| Hirudin (Heim et al., 1994) | 65 | 3 | 3 | | C-terminal degradation |
| A. australias insect-selective toxin 1 (Martin- | 69 | 4 | 6 | | low yield, perhaps due to |
| Eauclaire et al., 1994) | | | | | cleavage |
| insulin-like growth factor 1 (Bayne et al., | 70 | 3 | 6 | | full-length² |
| 1988) | | | | | |
| h parathyroid hormone¹ (Gabrielsen et al., | 84 | 0 | 14 | K26, | yield very low due to cleavage |
| 1990) | | | | F35, | |
| | | | | R44 | |

¹human
²No eendoproteolytic products reported.
³No proteotytic products found in Yap3, Mkc7 protease defective strain.

Some peptides less than 100 amino acids in length have been successfully expressed and secreted in yeast-based expression systems, however, the majority of these peptides are heavily constrained with disulfide bonds. Peptides with no disulfide bonds are difficult to express without significant proteolysis.

Of the 17 peptides listed in Table 1, 12 are constrained by disulfide bonds; of the remaining five, three were partially or completely cleaved by endoproteases, while the fourth, the antifreeze peptide AFP6, was only expressed as a multicopy form. The AFP6 peptide is also unusual in that it is 70% alanine and contains only two basic residues.

Glucagon was the only non-disulfide containing peptide, to our knowledge, that was expressed at significant levels (177 nM; Moody et al., 1987). Nonetheless, glucagon suffered some uncharacterized proteolysis during the identification of full-length glucagon. Moody et al. (1987) did not characterize two immunoreactive zones with a different RPLC retention time than full-length glucagon, but they did suggest that these fractions could be products of a cleavage at R¹⁷-R.

Examples 1-7 are discussed further in Copley et al. (1998, Biochem. J. 330:1333-1340), which is herein incorporated by reference in its entirety.

### Example 1

Preparation of Mas-DH+Gly Expression Vector.

Digan et al. (1992) isolated a cDNA encoding Mas-DH from pharate adult heads of Manduca sexta. Their cDNA clone encoded a 138 amino acid precursor of Mas-DH that included a putative signal sequence (amino acids 1-19) and a 40 amino acid pro-region followed by the Mas-DH coding region. There was a dibasic Kex2-like endoproteolytic site present on both ends of the coding region that would generate a precursor of natural Mas-DH, Mas-DH+Gly, upon processing by a Kex2-like enzyme.

We synthesized a synthetic Mas-DH gene that differed from the insect gene in that it was synthesized using the correct amino acid sequence for the Mas-DH+Gly peptide, but with the preferred codons for S. cerevisiae. Our synthetic gene did not include the native Mas-DH signal sequence nor the processing sites.

We began the construction of the synthetic gene by preparing a Mas-DH+Gly oligonucleotide (sense strand) that was 168 bases long and encoded a peptide linker containing a Kex2 protease cleavage site (Lys-Arg residues) preceded by four amino acids normally found prior to a Kex2 cleavage site in the α-factor precursor, followed immediately by the coding region for Mas-DH+Gly. Kex2 cleavage of the product of our gene at this site would produce Mas-DH+Gly with the mature N-terminus.

The resulting sequences are as follows:

| | | |
|---|---|---|
| Peptide linker (5′-3′): | | |
| Amino Acids: | Ala Asn Ser Gln Pro Met Tyr Lys Arg. | (SEQ ID NO.1) |
| Bases: | A GCG AAT TCT CAA CCA ATG TAC AAA AGA. | (SEQ ID NO.2) |
| Mas-DH + Gly (5′-3′): | | |
| Amino Acids: | Arg Met Pro Ser Leu Ser Ile Asp Leu Pro Met Ser Val Leu Arg | (SEQ ID NO.3) |
| | Gln Lys Leu Ser Leu Glu Lys Glu Arg Lys Val His Ala Leu Arg | |
| | Ala Ala Ala Asn Arg Asn Phe Leu Asn Asp Ile Gly. | |
| Bases: | AGA ATG CCA TCT TTA TCT ATT GAT TTA CCA ATG TCT | (SEQ ID NO.4) |
| | GTT TTG AGA CAA AAA TTA TCT TTG GAA AAA GAA | |
| | AGG AAA GTG CAT GCT TTG AGA GCT GCT GCT AAT | |
| | AGA AAT TTT TTG AAT GAT ATT GGT TAA | |
| | TGATCGATGCC. | |

Restriction sites were engineered into the sequence for ligation into the vector and gene manipulation. The synthetic oligonucleotide was purified by polyacrylamide gel electrophoresis (10% gel, 7.5 M urea). Primers complimentary to the 5′ and 3′ ends of the synthetic Mas-DH+Gly oligonucleotide were synthesized and used with the 168 nucleotide sense strand as the template to amplify the Mas-DH+Gly gene fragment using standard PCR techniques (94°, 1 min; 63°, 2 min; 72°, 2 min; 30 cycles and Taq DNA Polymerase (Perkin Elmer)).

The primer base sequences were as follows:

5′ Primer (5′-3′): AGCGAATTCTCAACCAATGT (SEQ ID NO. 5).

3′ Primer (5′-3′): GGCATCGATCATTAACCAAT (SEQ ID NO. 6).

The resulting double stranded DNA fragment containing the peptide linker plus the Mas-DH+Gly coding region was isolated by ligation into the pT7 Blue vector (Novagen®) and transformed into bacterial strain JM110 for amplification and sequencing. The DNA sequence was determined using standard methods (see, e.g., Sanger et al., 1977, which is hereby incorporated in its entirety).

After confirming the sequence, the gene was excised from pT7 Blue using EcoRl and Bsp106 I restriction enzymes (Stratagene®), and purified by agarose gel electrophoresis.

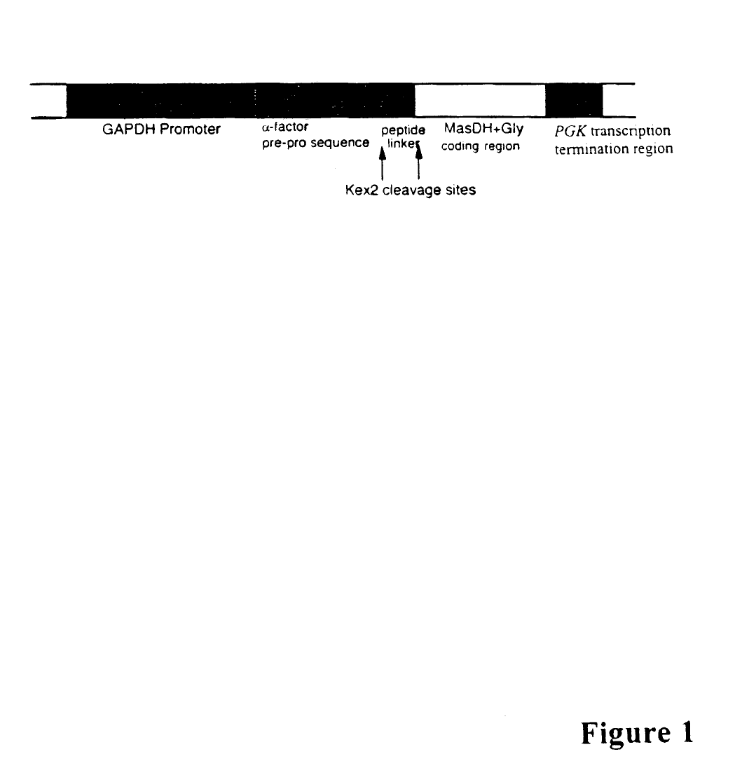

The purified fragment was then inserted into a yeast expression vector, YEp-IK (a 2μ plasmid-based E. coli-yeast shuttle vector which contains the TRP1 gene for selection in yeast) to create the plasmid pKSC2 (FIG. 1). While we chose to use this particular shuttle vector, the identity of the shuttle vector is not crucial to this invention. The fragment was ligated into the vector so that Mas-DH+Gly expression was controlled by the constitutive glyceraldehyde phosphate dehydrogenase (GAPDH) promoter for high-level expression of Mas-DH+Gly.

The synthetic Mas-DH+Gly fragment was ligated into the vector immediately after and in-frame with the α-factor pre-pro sequence, which would create an α-factor prepro-Mas-DH precursor that would be directed into and through the secretory pathway. After construction, the synthetic Mas-DH+Gly gene contained the GAPDH promoter, the α-factor prepro region, the peptide linker, the Mas-DH+Gly coding region, and finally the PGK transcription termination region (FIG. 1). Although the GAPDH promoter, and PGK transcription elements were used in our construction, it is expected that other such regulatory elements could be used that might vary to some degree the levels of mRNA for the Mas-DH precursor. Similarly, prepro regions other than that from the gene encoding α-factor have been successfully used in yeast. These elements are not critical for the invention presented here.

### Example 2

Expression of Mas-DH+Gly.

The pKSC2 plasmid was transformed into several different wild-type strains of S. cerevisiae, as well as a pep4 mutant strain which lacks numerous vacuolar protease activities, to optimize expression of the peptide (Table 2).

TABLE 2

| Strain† | Genotype | Plasmid* |
|---|---|---|
| CRY2 | MATα, can 1-100, ade 2-101, his3-11, 15, | pKSC2 |
| | leu2-3, 112, trp 1—1, ura3-1 | PKSC3 |
| HKY20 | CRY2 with yap3Δ::LEU2 | pKSC2 |
| | | PKSC3 |
| HKY21 | CRY2 with mkc7Δ::HIS3 | pKSC2 |
| | | PKSC3 |
| HKY24 | CRY2 with yap3Δ::LEU2, mkc7Δ::HIS3 | pKSC2 |
| | | PKSC3 |
| BFY106-4D | CRY2 with kex2-Δ2::HIS3 | pKSC2 |
| BFY25 | MATα, his3-Δ200, leu2-3, 112, trpl-Δ901, | pKSC2 |
| | ura3-52, ade5, pep4::LEU2 | PKSC3 |

*Indicates which plasmids were transformed into given strain

Transformation was done using a Li⁺ acetate-based method. Transformed cells were grown on synthetic media (SD medium containing 1.7 g/L yeast nitrogen base, Difco, without amino acids or (NH₄)₂SO₄, and supplemented with 4 g/L (NH₄)₂SO₄ and 20 g/L glucose). Amino acids and nucleotides were added to supplement auxotrophies, except Trp which was omitted to allow selection for cells retaining the plasmid. Cells were grown to saturation (˜24 h) at 30° C. in either polypropylene Nalgene® centrifuge bottles or polypropylene Erlenmeyer flasks coated with Sigmacote® to reduce adsorption of peptide to the vessel walls.

### Example 3

Purification of Mas-DH+Gly.

Solid Phase Extraction. The cells were removed by centrifugation (5,000 rpm, 10 min) from 2 L of culture. Soluble proteins from the supernatant were adsorbed on Vydac C₄ bulk protein support (20- to 30-μm particles) packed in a 75 ml polypropylene syringe barrel and equilibrated with 0.1% trifluoroacetic acid (TFA). The proteins were then eluted with 15% and 60% CH₃CN-0.1% TFA. 50 μl of a 1 mg/ml stock BSA (Sigma) solution was added to the 60% CH₃CN-0.1% TFA fraction. This fraction was further purified by an RPLC-based method.

RPLC Purification Method A Spectra Physics SP8700 pump, a Rheodyne loop injector, and a Spectra Chrom 100 variable wavelength detector set at 220 nm were used to purify large quantities of Mas-DH+Gly and the α-amidated Mas-DH. The pump was modified as follows: a low pressure slider valve (Rheodyne Model 5302) was placed before the pump in the liquid chromatograph to allow diversion of the incoming solvent to the pump, normally from the water reservoir, so that it was drawn from a polypropylene reservoir holding the diluted solution of peptide. It is possible to load hundreds of milliliters onto a 10 mm i.d. column with this modification.

The sample from solid phase extraction of 2 L of culture medium was in two aliquots of 40 ml. The 40 ml of 60% CH₃CN-0.1% TFA fraction from solid phase extraction was diluted to 10% CH₃CN-0.1% TFA with 200 ml 0.1% TFA and loaded into a 10 μm, 10 mm×250 mm Vydac C₄ semipreparative column equilibrated with 10% CH₃CN-0.1% TFA. Proteins were eluted at 5 ml/min using a linear gradient of 10% to 60% CH₃CN-0.1% TFA in 50 min. Peaks were collected by hand and 50 μl of 1 mg/ml BSA was added to each fraction. Fractions were analyzed by electrospray ionization mass spectrometry (ESI-MS) using a Finnigan MAT SSQ 710 mass spectrometer interfaced with an Analytica ESI ion source. Fractions corresponding to Mas-DH+Gly from the two RPLC runs were combined and dried to less than 1 ml final volume. This sample was then α-amidated to create Mas-DH.

### Example 4

Results of Attempted Expression.

Purification of the culture medium from wild type yeast cells using solid phase extraction followed by RPLC showed that there was substantial degradation of the Mas-DH+Gly product.

The primary products isolated were analyzed by ESI-MS and sequenced. The results showed that an endoproteolytic cleavage occurred between residues Lys²² and Glu²³. Two proteases are known to cleave proteins at basic residues in the yeast secretory pathway, Yap3 (Ledgerwood et al., 1996) and Mkc7 (Komano &Fuller, 1995). Accordingly we examined Mas-DH+Gly expression in a series of isogeneic strains that carried disruptions of the MKC7 and YAP3 genes (Table 3).

The plasmid pKSC2 was transformed into the single mutant strains HKY20 (yap3Δ) and HKY21 (mkc7Δ), the double mutant strain HKY24 (yap3Δ, mkc7Δ), along with the wild-type parent strain CRY2 (YAP3, MKC7). The plasmid was also transformed into the pep4Δ strain BFY25. Transformants were grown overnight under normal conditions to high cell density. The products in the culture medium were purified by solid phase extraction followed by RPLC.

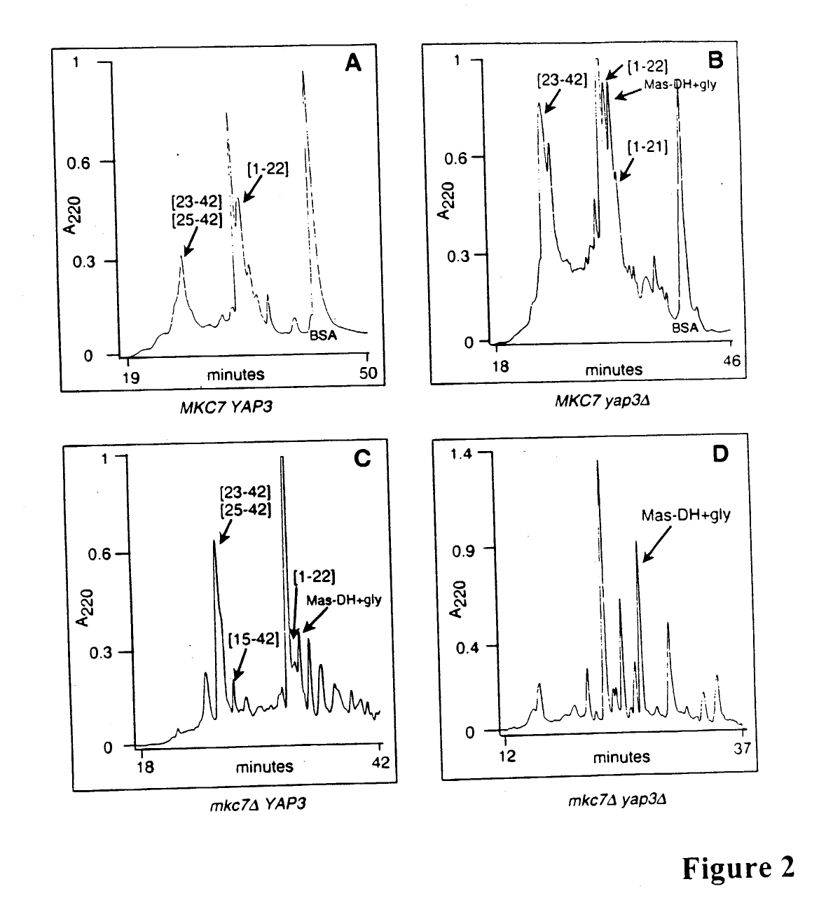

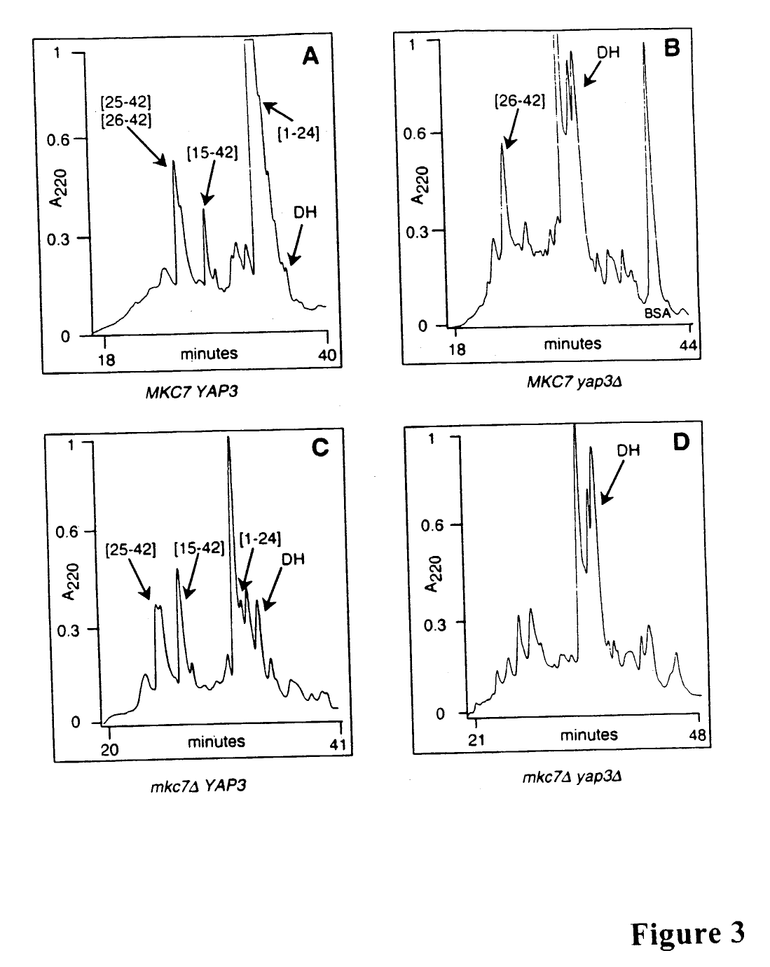

Mas-DH+Gly and peptide fragments were identified by RPLC retention time and ESI-MS (Table 3). The CRY2[pKSC2] wild-type strain produced the fragments Mas-DH[1-22], Mas-DH+Gly[23-42], and Mas-DH+Gly[25-42] (FIG. 2A). Full-length Mas-DH+Gly was not detected in this strain.

The same fragments were identified in the BFY25[pKSC2] (pep4Δ) strain even when the cells were grown in the presence of protease inhibitors. HKY20[pKSC2] (yap3Δ) produced a small amount of full-length Mas-DH+Gly (˜9% of recovered Mas-DH+Gly fragments). The major peptide components isolated from the media were the fragments Mas-DH[1-21], Mas-DH[1-22], and Mas-DH+Gly[23-42] (FIG. 2B). HKY21 [pKSC2] (mkc7Δ) produced a relatively larger amount of full-length Mas-DH+Gly (˜27% of recovered Mas-DH+Gly fragments) and yielded fragments Mas-DH[1-22], Mas-DH+Gly[23-42], and Mas-DH+Gly[16-42] (FIG. 2C). In all cases the fragment resulting from cleavage after Lys²² was the most abundant one found, suggesting that the Lys²² site is the primary cleavage site.

In the mkc7Δ deletion strain (HKY21[pKSC2]) there was evidence for a secondary cleavage site occurring after Arg¹⁵ (Table 3). The double mutant strain HKY24[pKSC2] (yap3Δ, mkc7Δ) produced ˜90% full-length Mas-DH+Gly and ˜10% “linker”-Mas-DH+Gly (containing a nonapeptide-linker, not removed by Kex2, but without the α-factor prepro sequence) (FIG. 2D). Thus both Yap3 and Mkc7 are capable of contributing to the cleavage of Mas-DH+Gly at the Lys²² residue, as well as at other sites.

TABLE 3
Proteolytic fragments of Mas-DH + Gly and Mas-DH[K22Q + Gly recovered after expression in S. cerevisiae

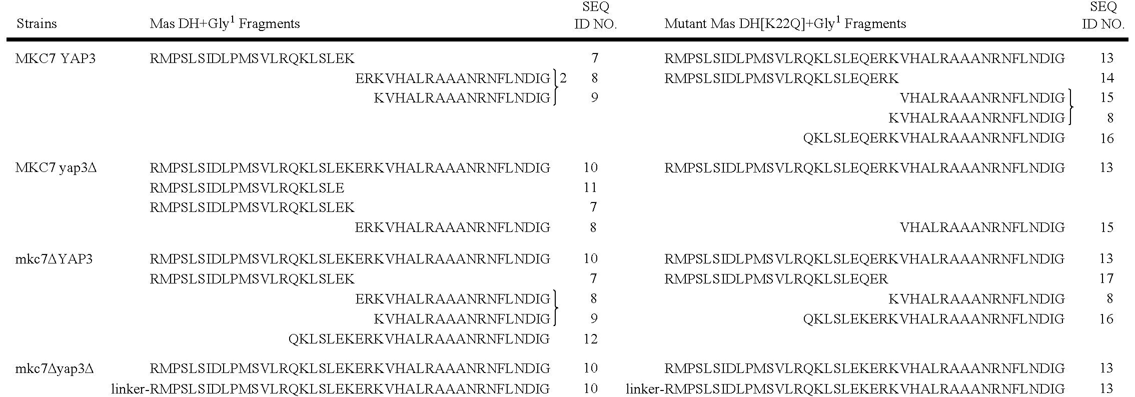

¹Mas-DH + Gly: RMPSLSIDLPMSVLRQKLSLEKERKVHALRAAANRNFLNDIG;
Mas-DH[K22Q] + Gly = RMPSLSIDLPMSVLRQKLSLEQERKVHALRAAANRNFLNDIG
²Fragments joined with a bracket on the right eluted together

### Example 5

Expression of a Mutant Form of Mas-DH+Gly; Mas-DH[22Q]+Gly.

Because cleavage at Lys²² by the Yap3 and Mkc7 proteases seemed to be responsible for the inability of wild-type yeast to produce Mas-DH+Gly, this Lys was changed to Gin. Substituting Gin for Lys at residue 22 should not affect the activity of the peptide because the change is conservative and in a location of the sequence that varies between the different diuretic hormones (alignment not shown).

Site-directed Mutagenesis. An oligonucleotide was synthesized (⁵′TCAAAGCATGCACTTTCCTTTCTTGTTCCAAAGATA³′) (SEQ ID NO. 14) that changed the Lys²² codon to one encoding Gin. This primer plus the 5′ Primer described above (see preparation of Mas-DH+Gly expression vector) were used to amplify the mutant 5′ portion of the gene using pKSC2 plasmid DNA (digested with HindIII) as the template (PCR conditions as stated above). The double-stranded product was purified by agarose gel electrophoresis (PCR Fragment 1).

To generate the full-length gene, PCR Fragment 1 was used as a primer along with the Mas-DH 3′ Primer and pKSC2 (digested with HindIII) as the template for a second PCR reaction using the same conditions as reported above. The product from this reaction (PCR 2 Product), was the full-length Mas-DH+Gly gene containing the Lys to Gin mutation and was purified using agarose gel electrophoresis. Because of a low yield of the PCR 2 Product, it was amplified by another round of PCR using the 5′ and 3′ Primers. The reamplified product containing the site-directed mutation was purified by agarose gel electrophoresis.

The mutant gene was digested with EcoR1 and Bsp106 I and ligated into the YEp-IK vector creating the plasmid pKSC3, which was transformed into E. coli (XL1-Blue, Stratagene®). To verify the construction of the mutant Mas-DH[M22Q]+Gly, pKSC3 was transformed into the BFY25 yeast strain. Yeast transformants were screened for expression of Mas-DH[K22Q]+Gly by RPLC purification and ESI-MS analysis (solid phase extraction and RPLC). The identity of Mas-DH[K22Q]+Gly was confirmed by amino acid analysis and Edman sequencing.

Plasmid pKSC3 carrying the K22Q mutant gene was transformed into the same set of strains used to express Mas-DH+Gly Each strain was grown overnight to saturation and components of the culture medium were purified by solid phase extraction and RPLC. Mas-DH[K22Q]+Gly and its fragments were identified by RPLC retention time and ESI-MS (Table 3). BFY25[pKSC3] produced primarily fragments of Mas-DH[K22Q]+Gly generated at now three cleavage sites; after Arg¹⁶, Arg²⁴ and Lys²⁵, while only ˜2% of the recovered Mas-DH[K22Q]+Gly was full-length peptide (FIG. 3A). Thus, elimination of the Lys²² cleavage by substitution did not prevent proteolysis, rather, it revealed secondary cleavage sites.

Using HKY20 and HKY21, it was possible to identify the protease responsible for each of the cleavages. Strain HKY20[pKSC3] (yap3Δ) produced the full-length Mas-DH[K22Q]+Gly (˜56% of total) and the fragment Mas-DH[K22Q]+Gly[25-42] (FIG. 3B) suggesting that Mkc7 cleaved after Lys²⁵. The mkc7Δ strain HKY21[pKSC3] generated full-length Mas-DH[K22Q]+Gly (˜25% of total) and the fragments Mas-DH[K22Q]+Gly[16-42] and Mas-DH[K22Q]+Gly[24-42] (FIG. 3C), suggesting that Yap3 cleaved after Arg at the two sites E R ⇓ K V and L R ⇓ Q K. This suggests that Mkc7 prefers to cleave after Lys and Yap3 after either Lys or Arg. Finally, the double mutant strain HKY24[pKSC3] (yap3Δ, mkc7Δ) produced full-length Mas-DH[K22Q]+Gly (˜90%) and “linker”-Mas-DH[K22Q]+Gly (˜10%) (FIG. 3D) demonstrating that proteolysis of Mas-DH[K22Q]+Gly was dependent on Yap3 and Mkc7.

### Example 6

Attempted use of Protease Inhibitors.

BFY25[pKSC2] cells were grown in the presence of a mixture of protease inhibitors, 10 mM DTT, and 1 M sorbitol to see if undesired proteolysis of Mas-DH+Gly could be inhibited.

The protease inhibitors used were 100 μM AEBSF, 100 μM pepstatin, 42 μM leupeptin, 1.5 μM aprotinin, 100 μM TLCK, and 400 μM 1,10-phenanthroline. The cells were grown using numerous combinations of the protease inhibitors listed above.

Full-length Mas-DH+Gly was not found in the supernatant after growth under any of these conditions.

### Example 7

Production of Biologically Active Mas-DH and Mas-DH[K22Q].

Mas-DH+Gly and Mas-DH[K22Q]+Gly expressed in the HKY24 strain (yap3Δ, mkc7Δ)were purified by solid phase extraction and RPLC, then α-amidated using the PAM enzyme.

Peptide α-amidation. The purified peptide from RPLC was α-amidated using the enzyme, peptidylglycine α-amidating monooxygenase (PAM, Unigene Laboratories). The reaction conditions were a buffer contained 0.03 M MES/NaOH pH 6, 5 μM CuSO₄, 0.01% Surfact-Amps X-100 (Pierce), 0.2 mM peptide, 0.1 mg/ml catalase (Sigma, from bovine liver), 1.5 mM ascorbate, and 8,000 units/ml PAM added in order, then incubated at 37° C. for 1 h. The α-amidated peptide was purified using RPLC. The 2 ml sample was diluted to 4 ml with 0.1% TFA and loaded into the column by loop injection. The peptide was eluted using a linear gradient of 10% to 60% CH₃CN-0.1% TFA in 25 min. The identity and purity of the peak was confirmed by ESI-MS.

The α-amidated Mas-DH and Mas-DH[K22Q] were then repurified. The α-amidated products were identified using ESI-MS. Before α-amidation an aliquot of the Mas-DH+Gly peptide was taken for amino acid analysis. The results indicated a yield of peptide of 0.8 mg/L (167 nM). After α-amidation and the final purification, the yield of Mas-DH was 0.4 mg/L (84 nM), while the final yield for the Mas-DH[K22Q] peptide was 0.13 mg/L (27 nM).

The purified amidated peptides were then tested for biological activity using a cAMP-based assay described previously to characterize Mas-DH isolated from insects.

cAMP Bioassay. Five newly emerged adult male M. sexta moths less than 8 h old were used. Malpighian tubules were dissected with care taken to cut tubules into 1 cm lengths and to use only the white portion of the tubules proximal to the midgut for assays.

The tubules were placed in a 96-well microtiter plate containing 100 μl saline (which contained the following constituents in millimolar concentration: 3, Na₂HPO₄; 5, MgCl₂; 1, CaCl₂; 5.8, KOH; 7.7, potassium citrate; 2.8, sodium succinate; 10, glucose; 3.6, alanine; 9.4, glutamine; 12.8, glycine; 9.7, histidine; 5.6, malic acid; 7.4, proline; 8.9, serine; 4.6, threonine;

180, sucrose; 5, NaHCO₃ and 0.5, isobutylmethylxanthine). It also contained 1 mg/ml of bovine serum albumin (Sigma). The saline was aerated with 95% O₂/5% CO₂ which adjusted the pH to 6.7).

The tubules were preincubated for one h at 30° C., then transferred to a polypropylene 96-well microtiter plate (Costar #3790) containing 100 μl serial dilutions of the peptide. EC₅₀ values for the recombinant peptides were calculated from the production of cAMP by Malpighian tubules.

To calculate the EC₅₀ value of synthetic Mas-DH and the two recombinant peptides, production of cAMP was measured for concentrations of peptide between 10⁻⁵ and 10⁻¹¹ M. Dried peptide was weighed and dissolved at an initial concentration of 10⁻⁵ M in MS-saline. This stock concentration was used to generate serial dilutions. Seven replicates were assayed for each concentration. The plate was incubated 1 h at 30° C.

After incubation, 50 μl aliquots from each well were transferred to a microcentrifuge tube for quantification of cAMP using the Gilman competitive binding assay performed as described by (Farndale et al., 1992), which is hereby included in its totality. The cAMP standard curve is linear in the range of 1 to 16 pmol. Samples incubated with high concentrations of peptide had to be diluted (10⁻⁵, 10⁻⁶ M; 1:10 dilution, 10⁻⁷, 10⁻⁸ M; 1:5 dilution) to remain within the linear region of the standard curve.

The recombinant peptides Mas-DH and Mas-DH[K22Q] had EC₅₀ values of ˜2 nM, equivalent to that of synthetic Mas-DH (Audsley et al., 1995), demonstrating that the Mas-DH synthesized in yeast is biologically active in an in vitro assay.

### Example 8

Additional Examples from Published Literature

The methods of the present invention are useful for the production of peptides such as insulin, human parathyroid hormone (hPTH), recombinant human albumin (rHA), glucagon, calcitonin, neuropeptide Y, but are not restricted to these peptides or their analogues.

For example, subsequent to the priority date of the present invention, the compositions and methods of the present invention have been utilized by others for the expression of proteins other than DH. Several examples are set forth in the following:

Recombinant Human Albumin. Kerry-Williams et al. (Jan. 30, 1998, Yeast 14(2):161-169) studied the expression of recombinant human albumin (rHA) in S. cerevisiae. While expression of rHA in normal yeast cells resulted in secretion of both mature albumin and a 45 kDa degradation production, a reduction in the amount of the degradation product was obtained by using yeast cells in which the YAP3 gene was disrupted.

Human Parathyroid Hormone (hPTH). Kang et al. (August 1998, Appl. Microbiol.

Biotechnol. 50(2):187-192) developed a human parathyroid hormone (hPTH) expression system using a host strain in which the gene for Yap3 was disrupted. They reported that “most of the hPTH secreted by the yap3 disruptant remained intact, whereas more than 90% of the hPTH secreted by the wild-type strain was cleaved” (Abstract). They concluded that “the yeast mutant lacking the Yap3 activity is a suitable host for the high-level expression of intact hPTH.”

Human beta-amyloid precursor protein. Komano et al. (Nov. 27, 1998, J. Biol. Chem. 273(48):31648-31651) reported that disruption of MKC7 and YAP3 in a vaculolar protease-deficient strain abolished human beta-amyloid precursor protein (APP) cleavage and release.

Glicentine Related Polypeptide and Glucagon. Brandt et al. (WO 9801535, published Jan. 15, 1998) disclose and claim methods for the production of short chain polypeptides in yeast using yeast strains having reduced activity of Yap3 protease. The authors assert that the disclosed methods can be used to increase the production of polypeptides such as glicentine related polypeptide (GRPP), glucagon, glucagon like peptide 1 (GLP-1), glucagon like peptide 2 (GLP-2), as well as various related truncated forms and analogues thereof The authors further assert that the production of such polypeptides is increased up to about 2-fold and even 10-fold compared to the yield from corresponding YAP3 wild-type yeast strains.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### ADDITIONAL REFERENCES

All references, articles, texts and patents referred to above and below are hereby incorporated by reference in their entirety.

Audsley, N., Kay, I., Hayes, T. K. & Coast, G. M. (1995) Comp. Biochem. Physiol. [A] 110A, 87-93.

Bayne, M. L., Applebaum, J., Chicchi, G. G., Hayes, N. S., Green, B. G. & Cascieri, M. A. (1988) Gene 66,235-244.

Bernasconi, P., Li, J. P., Reagan, J. D. & Kramer, S. J. (1992) Arch. Insect Biochem. Physiol. 21, 145-153.

Bitter, G. A., Chen, K. K., Banks, A. R. & Lai, P. H. (1984) Proc. Natl. Acad. Sci. USA 81, 5330-5334.

Bourbonnais, Y., Bolin, D. & Shields, D. (1988) J. Biol. Chem. 263, 15342-15347.

Digan, M. E., Roberts, D. N., Enderlin, F. E., Woodworth, A. R. & Kramer, S. J. (1992) Proc. Natl. Acad. Sci. USA 89, 11074-11078.

Driedonks, R. A., Toschka, H. Y., van Almkerk, J. W., Schaffers, I. M. & Verbakel, J. M. (1995) Yeast 11, 849-864.

Egel-Mitani, M., Flygenring, H. P. & Hansen, M. T. (1990) Yeast 6, 127-37.

Eipper, B. A., Stoffers, D. A. & Mains, R. E. (1992) Annu. Rev. Neurosci. 15, 57-85.

Farndale, R. W., Allan, L. M. & Martin, B. R. (1992) Adenylate cyclase and cAMP in Signal Transduction: A Practical Approach (Milligan, G., Eds.), pp 75-103, Oxford University Press, IRL Press, Oxford.

Gabrielsen, O. S., Reppe, S., Saether, O., Blingsmo, O. R., Sletten, K., Gordeladze, J. O., Høgset, A., Gautvik, V. T., Alestrom, P., Øyen, T. B. & Gautvik, K. M. (1990) Gene 90, 255-262.

Heim, J., Takabayashi, K., Meyhack, B., Marki, W. & Pohlig, G. (1994) European Journal of Biochemistry 226, 341-353.

Izumoto, Y., Sato, T., Yamamoto, T., Yoshida, N., Kikuchi, N., Ogawa, M. & Matsubara, K. (1987) Gene 59, 151-159.

Jacobson, M. A., Forma, F. M., Buenaga, R. F., Hoffman, K. J., Schultz, L. D., Gould, R. J. & Friedman, P. A. (1989) Gene 85, 511-516.

Komano, H. & Fuller, R. S. (1995) Proc. Natl. Acad. Sci. 92, 10752-10756.

Ledgerwood, E. C., Brennan, S. O., Cawley, N. X., Loh, Y. P. & George, P. M. (1996) FEBS letters 383, 67-71.

Martin-Eauclaire, M. F., Sogaard, M., Ramos, C., Cestele, s., Bougis, P. E. & Svensson, B. (1994) European J. Biochem. 223, 637-645.

Moody, A., Norris, F., Norris, K., Hansen, M. & Thim, L. (1987) FEBS Letters 212, 302-306.

Norris, K., Norris, F., Bjorn, S. E., Diers, I. & Peterson, L. C. (1990) Biol. Chem. Hoppe-Seyler 371, suppl 37-42.

Pang, S. Z., Oberhaus, S. M., Rasmussen, J. L., Knipple, D. C., Bloomquist, J. R., Dean, D. H., Bowman, K. D. & Sanford, J. C. (1992) Gene 116, 165-172.

Sanger, F., Nicklen, S. & Coulson, A. R. (1977) Proc. Natl. Acad. Sci. USA 74, 5463-5467.

Steiner, D. F., Smeekens, S. P., Ohagi, S. & Chan, S. J. (1992) J. Biol. Chem. 267, 23435-8.

Thim, L., Hansen, M. T. & Sorensen, A. R. (1987) FEBS Letters 212, 307-312.

Urdea, M. S., Merryweather, J. P., Mullenbach, G. T., Coit, D., Heberlein, U., Valenzuela, P. & Barr, P. J. (1983) Proc. Natl. Acad. Sci. USA 80, 7461-7465.

Zsebo, K. M., Lu, H. S., Fieschko, J. C., Goldstein, L., Davis, J., Duker, K., Suggs, S. V., Lai, P. H. & Bitter, G. A. (1986) J. Biol. Chem. 261, 5858-5865.

What is claimed is:

1. A method for the production of a polypeptide sensitive to proteolysis due to its content of arginine and lysine residues, wherein the method comprises the steps of:
(a) providing a yeast cell with reduced expression of either or both of the proteases encoded by YAP3 and MKC7;
(b) transforming the yeast cell of step (a) with a vector comprising a yeast promoter operably linked to a DNA sequence coding for the polypeptide sensitive to proteolysis;
(c) expressing the polypeptide; and
(d) isolating the polypeptide.

2. The method of claim 1 wherein the protease is encoded by YAP3.

3. The method of claim 1 wherein the protease is encoded by MKC7.

4. The method of claim 1 wherein the proteases are encoded by both YAP3 and MKC7.

5. The method of claim 1 wherein the reduced expression is due to an elimination of either or both of the proteases encoded by YAP3 and MKC7.

6. The method of claim 1 wherein the reduced expression is due to the disabling of either or both of the proteases encoded by YAP3 and MKC7.

7. The method of claim 1 wherein the reduced activity is due to antisense binding.

8. A method for the production of a polypeptide sensitive to proteolysis due to its content of arginine and lysine residues, wherein the method comprises the steps of:
(a) providing a yeast cell with reduced activity for either or both of the Yap3 and Mkc7 proteases;
(b) transforming the yeast cell of step (a) with a vector comprising a yeast promoter operably linked to a DNA sequence coding for the polypeptide sensitive to proteolysis;
(c) expressing the polypeptide; and
(d) isolating the polypeptide.

9. The method of claim 8 wherein the protease is the Yap3 protease.

10. The method of claim 8 wherein the protease is the Mkc7 protease.

11. The method of claim 8 wherein the proteases are both the Yap3 and Mkc7 proteases.

12. The method of claim 1 or claim 8 wherein the vector further comprises a signal peptide to direct secretion of the polypeptide from the yeast cell.

13. The method of claim 12 wherein the signal peptide is Saccharomyces cerevisiae α-factor prepro sequence.

14. The method of claim 1 or claim 10 wherein the polypeptide further comprises a glycine residue at its carboxyl terminus.

15. The method of claim 14 further comprising treating the polypeptide with peptidyl-glycine amidating enzyme after isolating the polypeptide.

16. The method of claim 1 or 8 wherein the polypeptide is selected from the group consisting of a precursor of Manduca sexta diuretic hormone, a site-directed mutant M. sexta diuretic hormone, albumin, human parathyroid hormone, insulin, glicentine related polypeptide, glucagon, calcitonin, neuropeptide Y, and analogues of each of these polypeptides.

17. A method for the production of a polypeptide sensitive to proteolysis due to its content of arginine and lysine residues, wherein the method comprises the steps of:
(a) providing a eukaryotic cell with reduced expression of either or both of the proteases encoded by YAP3 and MKC7;
(b) transforming the eukaryotic cell of step (a) with a vector comprising a promoter operably linked to a DNA sequence coding for the polypeptide sensitive to proteolysis;
(c) expressing the polypeptide; and
(d) isolating the polypeptide.

18. A method for the production of a polypeptide sensitive to proteolysis due to its content of arginine and lysine residues, wherein the method comprises the steps of:
(a) providing a eukaryotic cell with reduced activity for either or both of the Yap3 and Mkc7 proteases;
(b) transforming the eukaryotic cell of step (a) with a vector comprising a promoter operably linked to a DNA sequence coding for the polypeptide sensitive to proteolysis;
(c) expressing the polypeptide; and
(d) isolating the polypeptide.

19. The method of claim 17 or claim 18 wherein the eukaryotic cell is selected from the group consisting of an insect cell, a plant cell and a mammalian cell.

20. The method of claim 1, claim 8, claim 16 or claim 17 wherein the polypeptide is a heterologous polypeptide.


SEQUENCE LISTING (18 sequences)

SEQ ID NO: 1 - protein, 9 aa, Artificial Sequence
  Description of Artificial Sequence peptide linker
ANSQPMYKR

SEQ ID NO: 2 - DNA, 28 bp, Artificial Sequence
  Description of Artificial Sequence synthetic DNA encoding peptide linker
agcgaattctcaaccaatgtacaaaaga

SEQ ID NO: 3 - protein, 42 aa, Artificial Sequence
  Description of Artificial Sequence Mas-DH+Gly
RMPSLSIDLPMSVLRQKLSLEKERKVHALRAAANRNFLNDIG

SEQ ID NO: 4 - DNA, 140 bp, Artificial Sequence
  Description of Artificial Sequence synthetic DNA encoding Mas-DH+Gly
agaatgccatctttatctattgatttaccaatgtctgttttgagacaaaaattatctttggaaaaagaaaggaaagtgcatgctttgagagctgctgctaatagaaattttttgaatgatattggttaatgatcgatgcc

SEQ ID NO: 5 - DNA, 20 bp, Artificial Sequence
  Description of Artificial Sequence primer
agcgaattctcaaccaatgt

SEQ ID NO: 6 - DNA, 20 bp, Artificial Sequence
  Description of Artificial Sequence primer
ggcatcgatcattaaccaat

SEQ ID NO: 7 - protein, 22 aa, Saccharomyces cerevisiae
RMPSLSIDLPMSVLRQKLSLEK

SEQ ID NO: 8 - protein, 20 aa, Saccharomyces cerevisiae
ERKVHALRAAANRNFLNDIG

SEQ ID NO: 9 - protein, 18 aa, Saccharomyces cerevisiae
KVHALRAAANRNFLNDIG

SEQ ID NO: 10 - protein, 42 aa, Saccharomyces cerevisiae; identical to SEQ ID NO: 3

SEQ ID NO: 11 - protein, 21 aa, Saccharomyces cerevisiae
RMPSLSIDLPMSVLRQKLSLE

SEQ ID NO: 12 - protein, 27 aa, Saccharomyces cerevisiae
QKLSLEKERKVHALRAAANRNFLNDIG

SEQ ID NO: 13 - protein, 42 aa, Saccharomyces cerevisiae
RMPSLSIDLPMSVLRQKLSLEQERKVHALRAAANRNFLNDIG

SEQ ID NO: 14 - protein, 25 aa, Saccharomyces cerevisiae
RMPSLSIDLPMSVLRQKLSLEQERK

SEQ ID NO: 15 - protein, 17 aa, Saccharomyces cerevisiae
VHALRAAANRNFLNDIG

SEQ ID NO: 16 - protein, 27 aa, Saccharomyces cerevisiae
QKLSLEQERKVHALRAAANRNFLNDIG

SEQ ID NO: 17 - protein, 24 aa, Saccharomyces cerevisiae
RMPSLSIDLPMSVLRQKLSLEQER

SEQ ID NO: 18 - DNA, 36 bp, Artificial Sequence
  Description of Artificial Sequence synthesized oligonucleotide used for site-directed mutagenesis
tcaaagcatgcactttcctttcttgttccaaagata